(19) United States
(12) Patent Application Publication
(10) Pub. No.: US 2026/0364290 A1
(43) Pub. Date: Nov. 19, 2026

(54) ENDOTRACHEAL TUBES AND METHODS THEREOF

(71) Applicant: RESPAIR, INC., Pittsburgh, PA (US)

(72) Inventors: Carl SNYDERMAN, Pittsburgh, PA (US); Garrett Nicholas COYAN, Philadelphia, PA (US)

(21) Appl. No.: 19/146,024

(22) PCT Filed: Jan. 13, 2023
(86) PCT No.: PCT/US2023/010807

§ 371 Date: Jul. 7, 2025

(51) Int. Cl. A61M 16/04; A61M 16/00

(52) U.S. Cl. CPC A61M 16/0409; A61M 16/0003; A61M 16/0431; A61M 16/0463; A61M 16/0497; A61M 2016/0027; A61M 2205/0216; A61M 2205/0238; A61M 2205/32; A61M 2205/3327; A61M 2205/3331; A61M 2210/1032

(57) ABSTRACT

Disclosed herein are endotracheal tubes that have external baffles near the distal end of the tube to reduce unwanted migration of the tube relative to the trachea and to help prevent materials from passing between the outside of the tube and the tracheal walls. The baffles can be made of soft flexible material that conforms to and seals against the trachea. The baffles can be angled proximally or distally, or both. The baffles can be tapered in thickness and can vary in radial length. Some examples can include a supraglottic umbrella. Some examples can include an axially expandable portion along the tube. Some examples can include a loading tube that helps set the deflection orientation of the baffles within the trachea.

### FIELD

This application relates to endotracheal and similar tube devices for placement within hollow organs or cavities in the body.

### BACKGROUND

Conventional endotracheal tubes include an inflatable cuff to provide a seal for ventilation. Problems with such tubes include unwanted aspiration around the cuff resulting in ventilator-associated pneumonia, pressure necrosis from over-inflation of the cuff, unwanted tube migration (proximally and distally) with risk of hypoventilation or accidental extubation, and cuff malfunction (e.g., leakage). Ventilator-associated pneumonia is associated with significant morbidity and mortality and significantly increases the cost of care per episode.

Current endotracheal tubes suffer from additional deficiencies. For example, insertion of endotracheal tubes can produce friction with the glottis/trachea, making positioning difficult. Microbial biofilms also may form on the inserted endotracheal tube over time, increasing the risk of ventilator-associated pneumonia secondary to bacterial growth. Conventional endotracheal tubes are also combustible and pose a fire hazard in the presence of a heat source. Moreover, the position of a conventional endotracheal tube is difficult to monitor upon insertion and over time in intubated patients, which reduces the effectiveness of the endotracheal tubes and increases the intubation risk (e.g., aspiration and infection) for patients.

With respect to aspiration, secretions from the airway (e.g., supraglottic secretions) may be aspirated around portions of endotracheal tubes, which may come to rest and present a risk of further contamination of the airway. Endotracheal tubes with an additional lumen that leads to a suction port located 1-2 cm above the cuff have been developed, which are commonly referred to as Subglottic Suction Devices (SSD). Due to how inflatable balloons are manufactured and affixed to endotracheal tubes, a port 1-2 cm above the cuff is as close as it can get to pooled secretions. If the suction port was closer to the balloon cuff it would jeopardize the balloon's ability to maintain inflation.

Furthermore, the suction ports, as a simple lumen and port, are prone to clogging as secretions harden along the endotracheal tube. This can be mitigated by adding saline and “blowing” a high volume of air out of the port for a few seconds to dislodge clogs. However, the constraints of the port location make this a temporary fix as the port is still above the cuff although most fluids pool directly on the cuff. Without removing the fluids completely, there is a risk that the pooled fluid becomes laden with bacteria and seeps around the balloon sealing mechanism, causing infection.

### SUMMARY

Disclosed herein are endotracheal tubes that have external baffles near the distal end of the tube (e.g., in lieu of inflatable cuffs) to reduce unwanted migration of the tube relative to the trachea and to help prevent aspiration of materials between the outside of the tube and the tracheal walls. The baffles can be made of soft, flexible material that is resiliently deformable and that conforms to and seals against the tracheal walls. In some examples, the endotracheal tubes can include two or more sets of baffles, each set including differently shaped or oriented baffles. The baffles can be angled proximally and/or distally to provide biased properties in one or more directions. The baffles can be tapered in thickness and can vary in radial length. Some baffles can be helically coiled around the tube, wavy in cross-section, and/or concentric rings of varying diameter, and other configurations and permutations of the baffles can also be used as described and illustrated in more detail herein. Additionally, baffles of relatively soft material can be located at other portions of the endotracheal tubes of this technology, such as to act as a cushion or bumper to prevent contact with the mucosal surface.

Some endotracheal tubes of the examples described herein can include a supraglottic umbrella to further help prevent aspiration. Some examples can include an axially expandable portion along the tube to adjust the length between device features. Some examples can include a loading tube that helps set the deflection orientation of the baffles within the trachea and then is removed before the device is used. Some examples can include a plurality of flexible protuberances extending from the outside of the tube, either instead of or in addition to baffles. Some examples can include port systems that can be used as suction channels to remove materials from between, above, and/or below the baffles and/or delivery channels for medication delivery to the airway.

Some examples can include a forked distal end portion with each fork having its own baffles. Some examples can have baffles formed separately from the tubular portion, such as from different materials. Other examples can include an extra Murphy eye or ventilatory opening above a partial, non-occlusive baffle to facilitate continued ventilation of both lungs upon distal migration of the endotracheal tube into the mainstem bronchus.

In yet other examples of the endotracheal tubes disclosed herein, anti-fouling coatings can be applied to tubular portion(s) and/or the baffle(s) to prevent bacterial biofilm formation and ensure ease of tube insertion. In other examples, coatings can include anti-microbial agents and/or flame-retardant substances. Additionally, some examples include tubular portion(s) having differently-shaped cross-sections, including triangular and/or oval, for example, to limit the contact of the endotracheal tube with the tracheal wall or laryngeal cartilages, for example. The cross-sectional shape of the tubular portion of some examples can vary throughout the length according to the region at which the corre4sponding portion of the length is to be positioned.

To improve the positioning and/or location of endotracheal tubes and/or the baffles thereof, some examples of this technology include radiopaque markings on various surface(s). In other examples, sensors can be used to analyse positioning or other characteristics. In these examples, the sensors can be attached to the endotracheal tube to sense pressure, baffle deformation, pH, electrolyte levels, lactate levels, end-tidal CO2 or other ventilated gas content among other physiological parameters. Many other examples and permutations of the features described and illustrated herein can be used in accordance with the disclosed technology.

### BRIEF DESCRIPTION OF THE DRAWINGS

FIG. 1 shows a partial cross-section of a patient's airway with an exemplary endotracheal tube with baffles inserted into the trachea.

FIG. 2 shows the exemplary endotracheal tube with baffles of FIG. 1 in isolation.

FIG. 3 shows an exemplary endotracheal tube having perpendicular baffles.

FIG. 4 shows an exemplary endotracheal tube having distally angled baffles.

FIG. 5 shows an exemplary endotracheal tube having proximally angled baffles.

FIGS. 6A-B show an exemplary endotracheal tube having a unidirectional and bidirectional helical baffle, respectively.

FIG. 7 is a cross-sectional view of an exemplary endotracheal tube having tapered baffles.

FIG. 8 is a cross-sectional view of an exemplary endotracheal tube having baffles that vary in diameter.

FIG. 9 is a cross-sectional view of an exemplary endotracheal tube having baffles that each include both a proximally angled portion and a distally angled portion.

FIG. 10 is a cross-sectional view of an exemplary endotracheal tube having a helical baffle of varying diameter.

FIG. 11 is a cross-sectional view of an exemplary endotracheal tube having angled baffles.

FIG. 12 is a cross-sectional view of an exemplary endotracheal tube having one set of distally angled baffles and one set of proximally angled baffles, with the two sets tilted toward each other.

FIG. 13 shows an exemplary endotracheal tube having one set of distally angled baffles and one set of proximally angled baffles, with the two sets tilted away from each other.

FIGS. 14A-C show views of an exemplary endotracheal tube having one set of distally angled baffles and one set of proximally angled baffles, with the two sets tilted away from each other.

FIGS. 15A-B show views of an exemplary endotracheal tube having concentric connected baffles of distally increasing diameter.

FIG. 16 shows an exemplary endotracheal tube having a wavy baffle.

FIGS. 17A-C show an exemplary endotracheal tube having two sets of proximally angled baffles and insertion and retraction thereof.

FIGS. 18A-C show an exemplary endotracheal tube having a distal set of distally angled baffles and a proximal set of proximally angled baffles along with a loading tube and methods of use thereof.

FIGS. 19A-C show an exemplary endotracheal tube having perpendicular baffles and a supraglottic umbrella and insertion thereof.

FIG. 20 shows an exemplary endotracheal tube having two spaced apart sets of baffles and a supraglottic umbrella.

FIG. 21 shows an exemplary endotracheal tube having a set of perpendicular baffles, a supraglottic umbrella, and axially expandable baffles there-between.

FIG. 22 shows an exemplary endotracheal tube having flexible protuberances along the outer surface of the tube.

FIG. 23 is a cross-sectional view of the exemplary endotracheal tube of FIG. 22.

FIG. 24 shows an exemplary endotracheal tube having flexible protuberances along the outer surface of the tube.

FIG. 25 is a cross-sectional view of the exemplary endotracheal tube of FIG. 24.

FIG. 26 shows an exemplary endotracheal tube having a set of baffles with curled edges.

FIG. 27 shows an exemplary endotracheal tube having a set of broad baffles to seal the airway, a smaller baffle near a distal tip to prevent contact with the tracheal wall, and a proximal baffle at the subglottic region.

FIGS. 28A-B shows an exemplary endotracheal tube having a supraglottic umbrella positioned over the glottis.

FIGS. 29A-B show an exemplary endotracheal tube with a partial or complete projection in the subglottic region to prevent contact with the laryngeal mucosa.

FIG. 30 shows a non-circular cross-sectional profile for an exemplary endotracheal tube, with the posterior aspect having a flattened wall.

FIGS. 31A-B show exemplary endotracheal tube lumen shapes and cross-section profiles.

FIG. 32 is a cross-sectional view of an exemplary endotracheal tube having a non-circular baffle that has a flat posterior aspect.

FIG. 33 shows an exemplary endotracheal tube that includes a helical sensor embedded in the wall of the tube.

FIG. 34 shows an exemplary endotracheal tube that includes a set of baffles and sensors embedded in the baffles.

FIG. 35 shows an exemplary endotracheal tube that includes a series of axially spaced apart sensors along the tube.

FIGS. 36A-C show an exemplary endotracheal tube that includes radiopaque indicators.

FIGS. 37A-B show an exemplary endotracheal tube with proximal and distal lateral openings

FIGS. 38A-D show exemplary endotracheal tubes with exemplary port systems for suction and/or medication delivery.

### DETAILED DESCRIPTION

Disclosed herein in some examples are endotracheal tubes that include external circumferential baffles near a distal end of the tube, which provide a seal for ventilation, reduce unwanted motion of the tube relative to the trachea, and restrict materials from passing between the outside of the tube and the tracheal walls (e.g., aspiration). The baffles can replace and improve the functionality of a conventional inflatable cuff at the distal end of the tube. In some examples, baffles can be included in addition to one or more inflatable cuffs. As described herein, the disclosed baffles in some examples can have many different shapes, sizes, placements, orientations, groupings, and/or numbers. The baffles in some examples can comprise a series of soft, flexible, annular membranes that extend outwardly from the outer surface of the tube and provide circumferential contact with the tracheal mucosa, helping to prevent aspiration and migration while sealing the airway for ventilation.

In some examples, additional features can be included, such as a supraglottic umbrella proximal to the baffles and/or a loading tube to help orient the baffles properly when inserted into the trachea. The examples disclosed herein can include any one or more of the following features, and/or other features described and illustrate in more detail below: multiple circumferential baffles that line the exterior surface of the endotracheal tube, wherein the baffles are soft and pliable so that they do not hinder tube insertion; multiple baffles that provide adequate seal of airway for positive pressure ventilation; pliable baffles that allow minimal escape of air with positive pressure ventilation that prevents pooling of secretions and aspiration; angulation of baffles that resists movement of the tube; multiple groups of baffles with different angulation that resists distal and proximal movement of the tube; varied lengths of groups of baffles that allows “anchoring” of the tube; proximal or distal suction channels; pressure sensors on baffle(s) that detect movement of the tube, orientation of the baffles, and/or degree of bending/flattening of the baffles; proximal umbrella that secures the tube above the supraglottis; proximal umbrella that prevents aspiration of gastric contents and directs pharyngeal contents toward the esophagus; translucent materials that improve visualization; and/or sterilizable and re-usable tubes or components thereof.

One or more of the examples described and illustrated herein can provide any one or more of the following advantages over conventional endotracheal tubes and/or other advantages explained in more detail below: absence of an inflatable cuff, which avoids cuff-related morbidity and malfunction; more effective at preventing aspiration, which is the major cause of ventilator-associated pneumonia; decreased risk of tube migration with associated morbidity; easier to use with minimal learning curve; improved adoption of use; decreased production costs compared to expensive modified tubes and monitoring devices; decreased monitoring of cuff pressures and tube position by healthcare personnel; suitable for pediatric patients; sterilizable and re-usable for resource-poor environments; resists movement from high ventilation pressures; and/or allows escape of high ventilation pressures.

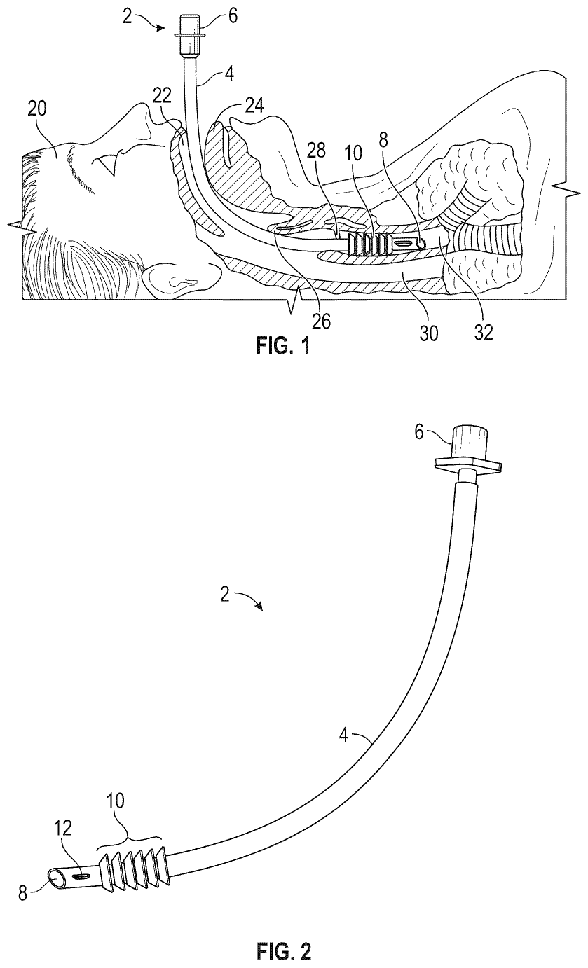

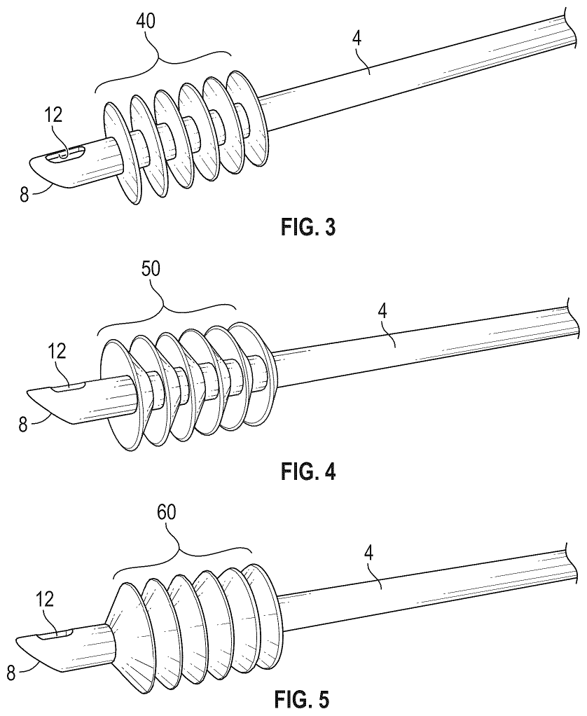

Referring now to FIG. 1, a partial cross-section of a patient 20 illustrating the airway with an exemplary endotracheal tube 2 with baffles 10 inserted into the trachea 32 is illustrated. FIG. 2 shows the endotracheal tube 2 in isolation. The endotracheal tube 2 can comprise a tubular portion 4, an inlet adaptor 6 at a proximal end of the tubular portion 4 for coupling to a ventilator or other device, a distal outlet 8, a lateral opening 12, and baffles 10, which are annular and positioned around a distal portion of the tubular portion 4 in this example.

As shown in FIG. 1, the endotracheal tube 2 is inserted through the mouth 22, over the tongue 24, past the epiglottis 26, through the larynx 28, and into the trachea 32. The esophagus 30 is also shown. The baffles 10 are soft and flexible and conform to the shape of the trachea 32 at a location between the larynx and the bronchi. The baffles 10 gently engage the walls of the trachea 32 to hold the endotracheal tube 2 in the desired position, such as with the distal outlet 8 proximal to the bronchi. In some examples, the endotracheal tube 2 prevents distal migration into one bronchus and avoids obstruction of the other bronchus, thereby maintaining ventilation of both lungs. The baffles 10 create a seal to support ventilation while substantially blocking aspiration between the endotracheal tube 2 and the trachea 32 and resisting unwanted axial movement of the endotracheal tube 2.

The baffles 10 can be soft and flexible enough to allow proximal release of air past the baffles 10 during high positive pressure in the lungs during ventilation. The softness of the baffles 10 and the distribution of the baffles 10 along the tubular portion 4 can also help more evenly distribute pressure across a larger area and reduce the risk of pressure necrosis of the tracheal mucosa.

In any of the examples described herein, the baffles 10 can include pressure sensors to provide pressure data to a monitoring device, e.g. via wires running along the tubular portion 4 and/or via wireless connections. Such sensors can be used to detect excess pressure or insufficient pressure, and to detect migration of the endotracheal tube 2.

In any of the examples described herein, the endotracheal tube 2 can include integrated suction channels, conduits, or catheters running along the tubular portion 4 and coupled to openings in the walls between or adjacent to the baffles 10. Such suction conduits can be used to suck fluids or other materials that build up between or adjacent to the baffles 10. For example, suction openings can be positioned between each adjacent pair of baffles 10, just above the most proximal baffle of each set of baffles 10, and/or just below the most distal baffle of each set of baffles 10. Removing excess materials around the baffles 10 can help prevent unwanted aspiration and infections.

In any of the examples described herein, the tubular portion 4 can include reinforcing members, such as wires, bars, coils, rings, etc., to help prevent kinking, compression, or other unwanted deformation of the tubular portion 4. In some examples, the baffles can also include reinforcing members.

In some examples, the tubular portion 4 can include a double lumen that forks into two distal end portions, or forks, for independent ventilation of each lung. Each fork can terminate in or adjacent to a respective one of the bronchi. In such examples, each fork can have its own set of baffles 10. In some examples, a larger, proximal set of baffles 10 is located around the tubular portion 4 before the fork, and one or more smaller sets of baffles 10 are located around each fork.

In one particular example, the tubular portion 4 includes first and second forks and first and second sets of baffles 10. In this example, the second fork terminates distal to the first fork and the second set of baffles 10 is disposed distal to the first set of baffles 10. More specifically, the second fork is configured to be positioned in a single bronchus along with the second set of baffles 10 proximal to the distal end of the second fork. The first set of baffles 10 is configured to be positioned in the trachea with the distal end of the first fork disposed between the first and second sets of baffles 10. In other words, the first set of baffles 10 is located proximal to the distal end of the first fork. Other configurations of multiple lumens and/or forks for the tubular portion 4, and/or multiple sets of baffles 10, can also be used in other examples.

In some examples, the tubular portion 4 and one or more of the set of baffles 10 can be separately formed and subsequently coupled together (e.g., via adhesion or another bonding method). Separate formation allows the baffles 10 and the tubular portion 4 to optionally be constructed of different materials having different properties, such as density, thickness, chemical composition, mechanical characteristics (e.g., stiffness), for example. In one particular example, the baffles 10 are formed together as a monolithic structure having a lumen and subsequently coupled to the previously-formed tubular portion 4, which is inserted through the lumen until the baffles 10 are positioned in a desired location for attachment. In other examples, each baffle of the baffles 10 is formed separate from the tubular portion 4 and is subsequently coupled to the tubular portion, and other methods for constructing the endotracheal tube 2 can also be used in other examples.

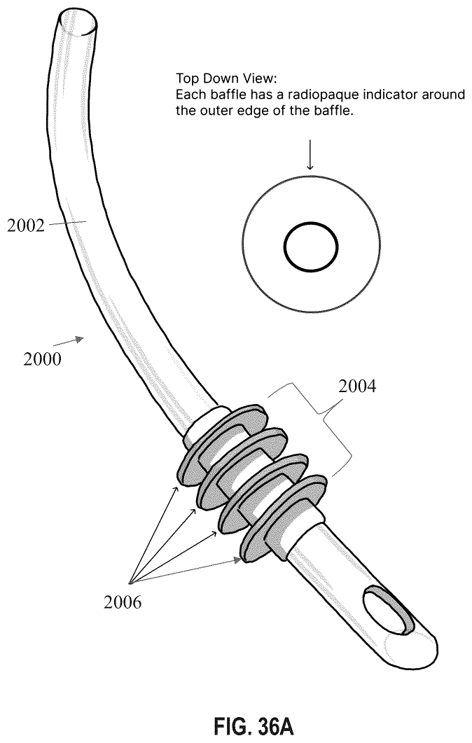

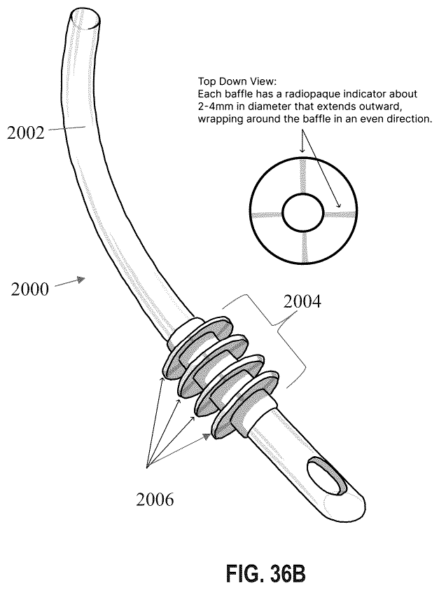

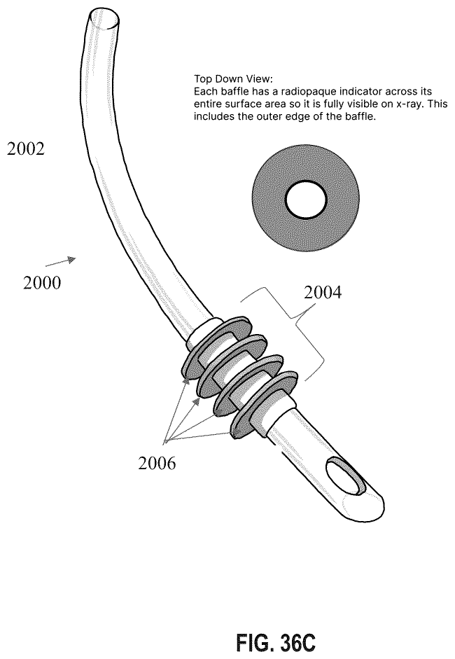

In some examples, the tubular portion 4 and/or the baffles 10 can be made of a same material with variable thickness, and therefore variable rigidity and flexibility. In other examples, the baffles 10 and the tubular portion 4 can be formed of different materials that have different properties. In some examples, the tubular portion 4 and/or the baffles 10 can include a composite construction, being constructed of a combination of two or more different materials. Such materials can include various polymeric materials, metallic/alloy materials, reinforcing components, outer coatings, radiographic elements, visual markings, etc. Endotracheal tubes of this technology with radiologic markers are described and illustrated in more detail below with reference to FIGS. 36A-B.

In some examples, the baffles 10 and/or an outer surface of the tubular portion 4, for example, can be coated or impregnated with one or more substances. The substance(s) may be applied post-manufacturing of the endotracheal tube 2 and/or baffles 10, in addition to, or in place of, being added to one or more components (e.g., the baffles 10 and/or the tubular portion 3) of the endotracheal tube 2 itself during manufacturing. Accordingly, the substances can be applied post-endotracheal tube 2 production or introduced/mixed in as part of the endotracheal tube 2 manufacturing process.

In some particular examples, the substance(s) can include an anti-fouling coating that can be applied to, or impregnated within, the endotracheal tube 2 and/or the baffles 10, for example, to prevent bacterial biofilm formation and ensure ease of endotracheal tube insertion. In these examples, the anti-fouling coating can reduce the risk of a ventilator-associated pneumonia secondary to bacterial colonization as a result of microbial biofilm formation on the endotracheal tube 2 or a portion thereof.

In other examples, the substance(s) can include a flame-retardant to mitigate the fire hazard of the endotracheal tube 2 in the presence of a heat source (e.g., laser or electrocautery). In yet other examples, the substance(s) can include a non-stick or anti-stick material, which can reduce the friction with the glottis/trachea caused by the endotracheal tube 2 and/or baffles 10 and facilitate easier positioning of the endotracheal tube 2. Other types and/or number of substances can also be used in other examples. Accordingly, any number of different substances, and/or combinations of substances, can be used for any number of different components (e.g., baffles 10 and/or tubular portion 4) of the endotracheal tube 2 in any of the examples described and illustrated herein.

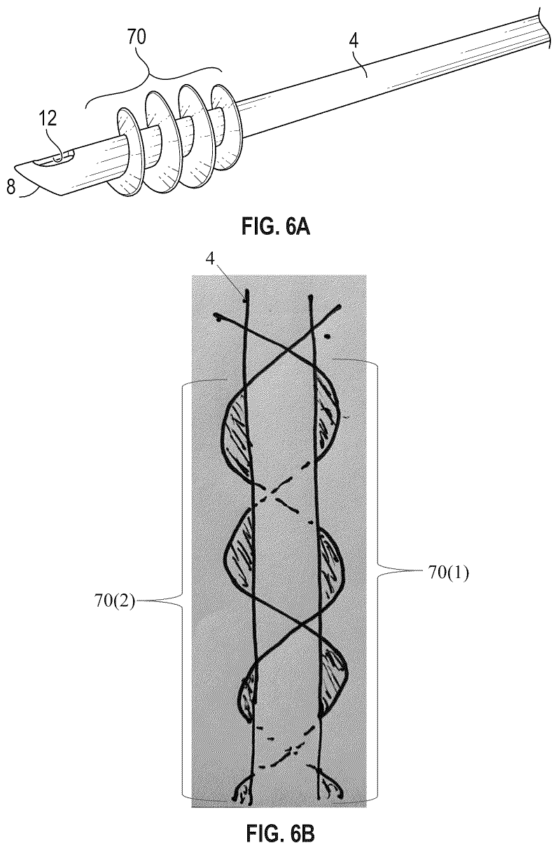

Referring to FIGS. 3-12, various different types of exemplary baffles, any of which can be used in a manner similar to FIG. 1, are illustrated. Referring more specifically to FIG. 3, an example having thin, disk-shaped baffles 40 that extend perpendicularly from the tubular portion 4 is illustrated.

FIG. 4 shows an example having distally angled baffles 50 that have a frustoconical shape. The distally angled baffles 50 can be more resistant to unwanted distal migration, while allowing the endotracheal tube 2 to be moved proximally (e.g., removed) with less resistance.

FIG. 5 shows an example having proximally angled baffles 60 (similar to baffles 10) that also have a frustoconical shape but facing the opposite direction. The proximally angled baffles 60 can be more resistant to unwanted proximal migration (e.g., accidental extubation), while allowing the endotracheal tube 2 to be moved distally (e.g., insertion) with less resistance.

FIG. 6A shows an example having a helical baffle 70 that coils around the tubular portion 4. The helical baffle 70 can also vary in diameter, such as having a smaller diameter at the ends and larger diameter in the middle. A helical baffle can have the unique property of a continuous helical pathway between the turns of the baffle that can allow secretions and other materials to pass through the baffle without stasis, which can help avoid unwanted build-up around the helical baffle 70. The helical baffle 70 can also help avoid focal circumferential pressure along an annular ring section of the trachea. The helical baffle 70 can also provide enhanced resistance to torsional/rotational motion of the tube with the trachea.

FIG. 6B shows another example having two helical baffles 70(1)-70(2) in a bidirectional helical baffle configuration. Since circumferential circular and other types of baffles may cause ischemia with circumferential scarring and/or stenosis, the bidirectional helical baffle with the two helical baffles 70(1)-70(2) in this example advantageously decreases the risk of circumferential scarring and/or stenosis while still providing airway protection. The two helical baffles 70(1)-70(2) are advantageously located so as to contact alternating portions of the tracheal wall Another number of helical baffles in a different configuration (e.g., with portions having disparate diameters and/or thicknesses) can also be used in other examples.

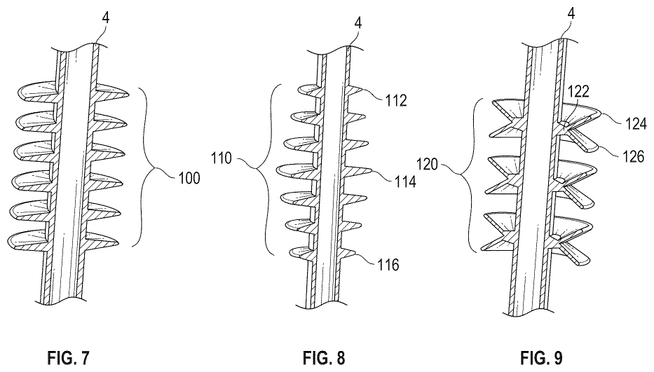

FIG. 7 is a cross-sectional view of an example having equal diameter tapered baffles 100 that are thicker adjacent to the tubular portion 4 and gradually become thinner moving radially outward, providing a flexible edge and that is non-traumatic to the tracheal mucosa. The equal diameter tapered baffles 100 are equal in diameter in this particular example.

FIG. 8 is a cross-sectional view of an example having tapered baffles 110 that vary in diameter. The baffles 112 and 116 toward the proximal and distal ends, respectively, have smaller diameters while the middle baffles 114 have larger diameters. Such varying diameter tapered baffles 110 can provide a better fit where the trachea has a non-constant inner diameter, such as a bulge or narrowing.

FIG. 9 is a cross-sectional view of an example having baffles 120 that each include both a proximally angled portion 124 and a distally angled portion 126 extending from an inner base portion 122. Having a proximally angled portion 124 and a distally angled portion 126 that are angled in both directions can provide better sealing and migration resistance in both directions.

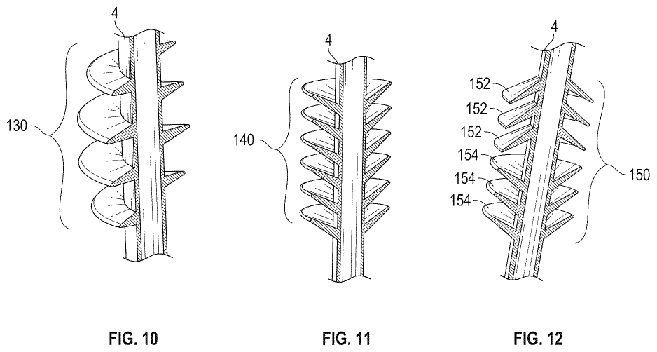

FIG. 10 is a cross-sectional view of an example having a tapered, helical baffle 130 of varying radius, with a larger radius in the middle and smaller radii at the ends.

FIG. 11 is a cross-sectional view of an example having tapered, proximally angled baffles 140.

FIG. 12 is a cross-sectional view of an example having tapered baffles 150 comprising one set of proximally angled baffles 152 and one set of distally angled baffles 154, with the two sets tilted toward each other. Having proximally angled baffles 152 and distally angled baffles 154 that are angled in both directions can provide better sealing and migration resistance in both directions.

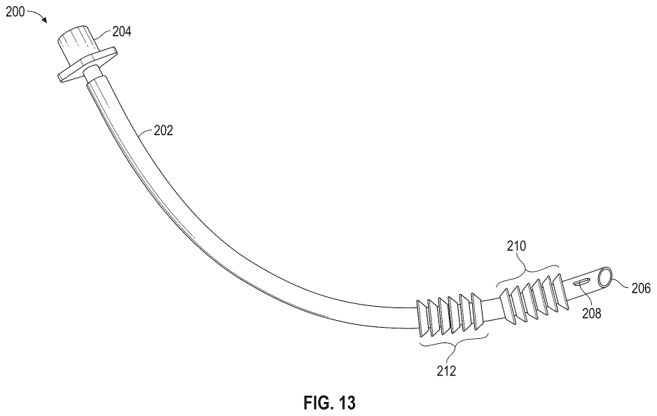

FIG. 13 shows an exemplary endotracheal tube 200 comprising a tubular portion 202, proximal adaptor 204, distal opening 206, lateral opening 208, a set of distally angled baffles 210, and a set of proximally angled baffles 212, with the two sets tilted away from each other. The endotracheal tube 200 is illustrated with a gap or spacing between the two sets of angled baffles 210 and 212, which can vary in length from zero to several centimeters. Similarly, the number of baffles in each of the two sets of angled baffles 210 and 212 can vary from one to six, or more.

Analogously, any of the examples disclosed herein can have any number of baffles in each set of baffles, and those examples with multiple sets of baffles can have any spacing between the sets. Furthermore, the diameter of the baffles in any example can vary from just greater than the diameter of the tubular portion (e.g., small annular ribs) to several centimeters greater than the diameter of the tubular portion.

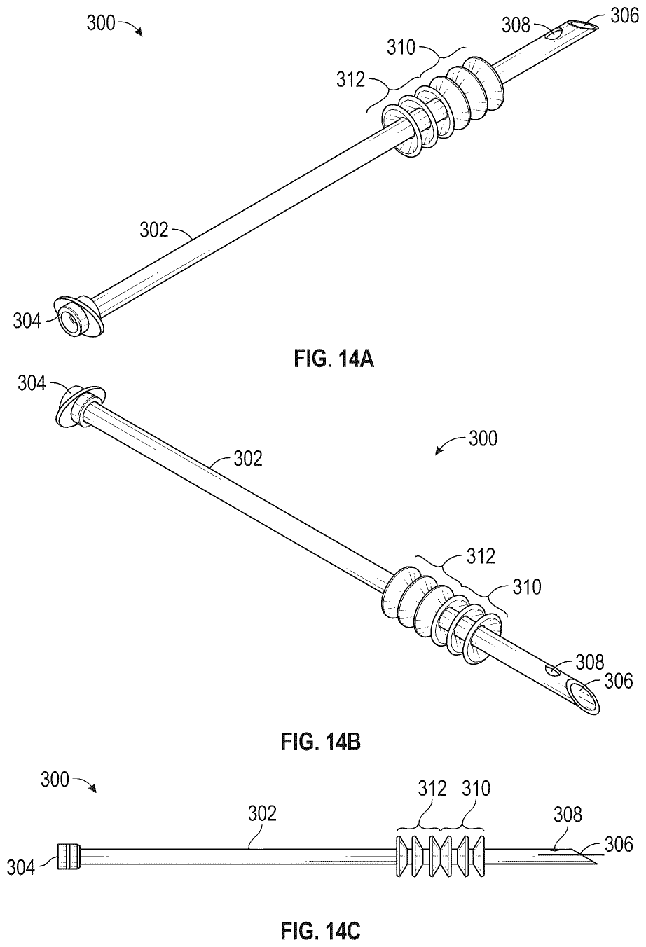

FIGS. 14A-C show views of another exemplary endotracheal tube 300 having a tubular portion 302, a proximal adaptor 304, a distal opening 306, a lateral opening 308, a set of distally angled baffles 310, and a set of proximally angled baffles 312, with the two sets of angled baffles 310 and 312 positioned relatively close together and tilted away from each other.

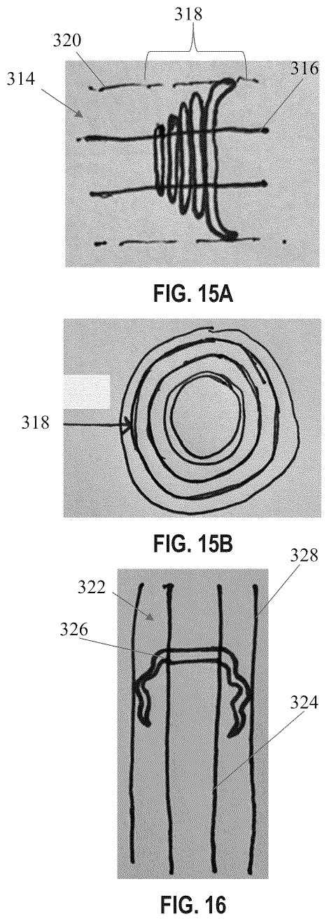

FIGS. 15A-B show views of an exemplary endotracheal tube 314 having a tubular portion 316 and concentric connected baffles 318 of distally increasing diameter disposed proximate (e.g., attached to) the tubular portion 316. The concentric connected baffles 318 in this example can facilitate improved sealing across a wider range of sizes (i.e., diameters) of the trachea 320. More specifically, the concentric connected baffles 318 in this example include a plurality of baffles including a most proximal baffle having a smallest diameter and a most distal baffle having a largest diameter, with the intermediate baffles having distally increasing diameters.

Accordingly, the one of the concentric connected baffles 318 that is of optimal diameter (e.g., substantially corresponding to the diameter of the trachea 320) will provide a sealing interface with the trachea 320, and any one or more of the concentric connected baffles 318 having a larger diameter may fold toward a distal portion of the endotracheal tube 314. While five baffles are illustrated in FIGS. 15A-B, any number of baffles could be used in other examples, including as few as two baffles. Additionally, the concentric connected baffles 318 are illustrated as concentric connected rings, but other shapes can be used and/or the baffles can be disconnected (i.e., separated along the tubular portion 316) in other examples. Thus, the endotracheal tube 314 illustrated in FIGS. 15A-B advantageously maintains a seal across a wide range of tracheal sizes.

Referring to FIG. 16, an exemplary endotracheal tube 322 having a tubular portion 324 and a wavy baffle 326 disposed proximate (e.g., attached to) the tubular portion 324 is illustrated. While only one wavy baffle 326 is illustrated in this example, any number of wavy baffles could be used in other examples, each having the same or different characteristics (e.g., diameter) and/or profile. The wavy baffle 326 in this example includes curved or rounded portion(s) protruding distally and/or proximally that are configured to interface with the trachea 328 and fold distally to thereby form a seal.

Thus, the rounded surface of contact for the wavy baffle 326 can be disposed at an optimal location (e.g., diameter) based on the diameter of the trachea 328, with any additional outer portion of the wavy baffle 326 configured to fold distally. Accordingly, as in the examples described and illustrated above with reference to FIGS. 15A-B, the example illustrated in FIG. 16 also advantageously maintains a seal across a wider range of tracheal sizes.

Optionally, baffle mechanics (e.g., folding) can be improved in some examples by forming the baffle(s) of any of the examples described and illustrated herein with multiple materials having different properties. Accordingly, while the diameter, thickness, profile, and/or shape, among other characteristics, may vary for any portion(s) of any particular baffle, or across any number of baffles in a same baffle set, in some examples the varying characteristics are formed using multiple materials with different properties.

For example, baffle(s) may be formed of a material that is relatively stiff in a proximal portion and relatively pliable in a distal portion that is configured to contact the tracheal wall. In another example, baffle(s) may be formed of a material that is relatively stiff in an interior portion disposed toward the tubular portion of an endotracheal tube and relatively pliable in an exterior portion disposed away from the tubular portion and configured to interface with the tracheal wall. Any other permutations of materials having any number of different properties can be used in other examples to form a particular baffle or set of baffles in any of the examples described and illustrated herein.

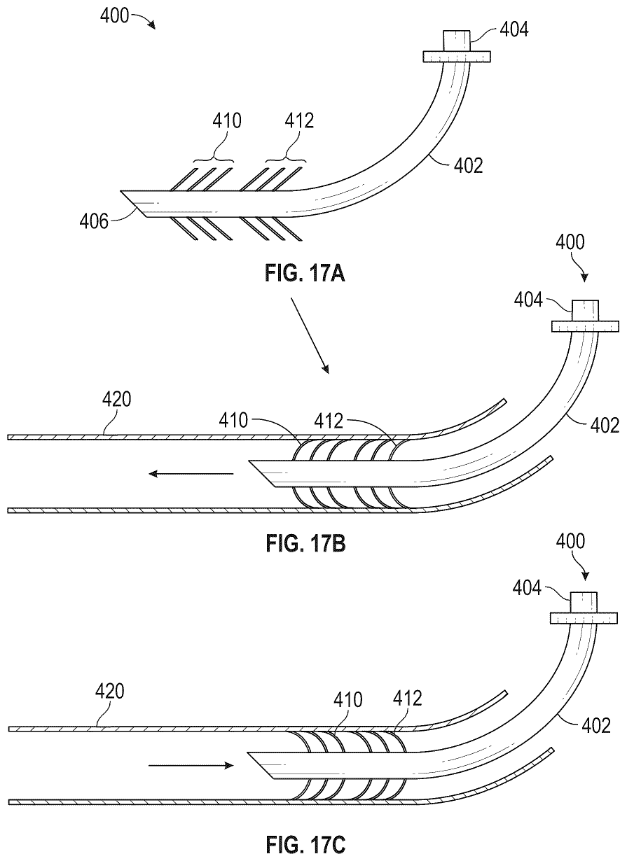

FIG. 17A schematically illustrates an exemplary endotracheal tube 400 having a tubular portion 402, proximal adaptor 404, distal opening 406, and two sets of proximally angled annular baffles 410 and 412 (each similar to the baffles 60). FIG. 17B illustrates insertion of the endotracheal tube 400 into a trachea 420, which causes the baffles 410 and 412 to defect proximally as they contact the inner surface of the trachea. The diameter of the proximally angled annular baffles 410 and 412 is larger than the inner diameter of the trachea, forcing the flexible proximally angled annular baffles 410 and 412 to deform further proximally as the endotracheal tube 400 moves distally.

Conversely, FIG. 17C illustrates how retracting the endotracheal tube 400 proximally relative to the trachea 420 causes the baffles to defect the other direction, distally, due to their engagement with the trachea. This phenomenon can be employed beneficially when placing any of the endotracheal tubes disclosed herein in a patient, as small adjustments of the endotracheal tube either proximally or distally within the trachea can re-orient the baffles in different directions, allowing a user to select whichever baffle orientation is more desirable for a given patient situation, and re-orient the baffles at any time without having to remove the endotracheal tube from the trachea.

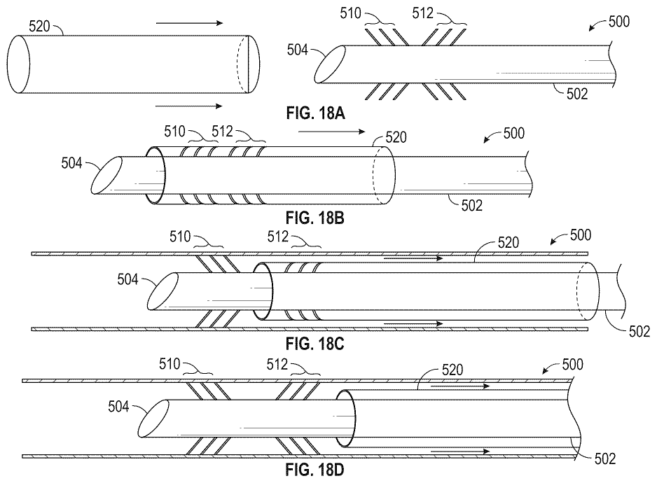

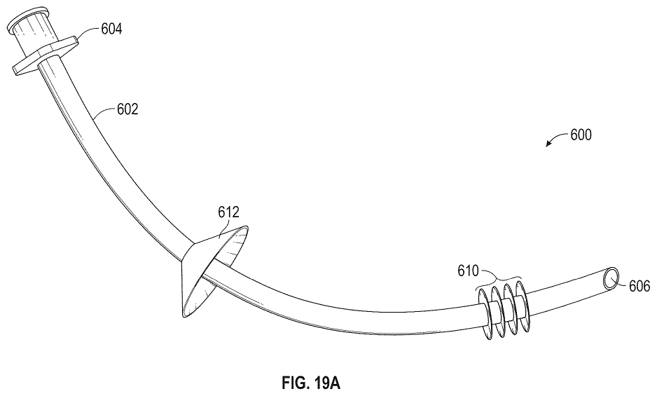

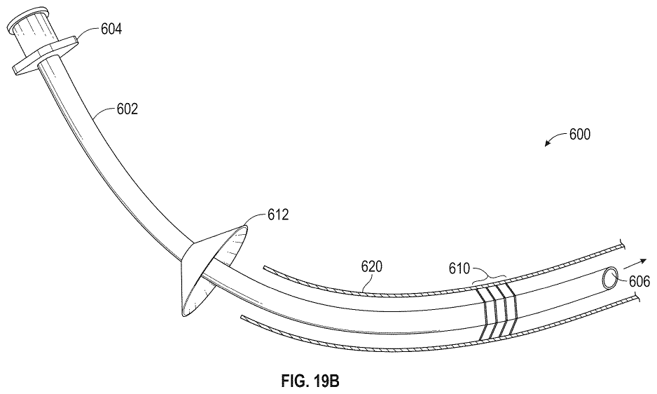

FIGS. 18A-18D illustrate how an additional loading tube 520 can be used with any of the endotracheal tubes disclosed herein to better control the orientation of the baffles within the trachea. Without using such a loading tube 520, all of the baffles may end up angled in the same direction within the trachea. FIG. 18A shows an exemplary endotracheal tube 500 having a tubular portion 502, a distal opening 504, a distal set of distally angled baffles 510, and a proximal set of proximally angled baffles 512, along with a loading tube 520. The loading tube 520 can be passed over the baffles prior to insertion of the device into the patient, as shown in FIG. 18A. The endotracheal tube 500 with the loading tube 520 covering the baffles can then be inserted into to the trachea.

As shown in FIG. 18C, the loading tube 520 can then be partially retracted proximally relative to the endotracheal tube 500 and the trachea, uncovering the distal set of distally angled baffles 510 and allowing them to resiliently unfurl into contact with the trachea. In order to then ensure the distal set of distally angled baffles 510 are angled distally (as shown in FIG. 18C), the endotracheal tube 500 and loading tube 520 can be moved proximally together relative to the trachea a small distance. Then, with the distal set of distally angled baffles 510 desirably oriented, the loading tube 520 can be moved further proximally while the endotracheal tube 500 is held steady relative to the trachea (as shown in FIG. 18D) to release the proximal baffles from the loading tube 520. Using this method, the endotracheal tube 500 (or other multi-baffle example disclosed herein) can be deployed in the trachea with one set of baffles angled distally and another set of baffles angled proximally.

FIG. 19A shows an exemplary endotracheal tube 600 including a tubular portion 602, proximal adaptor 604, distal opening 606, a set of baffles 610, and a supraglottic umbrella 612 positioned around the tubular portion 602 proximal to the set baffles 610. The supraglottic umbrella 612 can be positioned over the supraglottic larynx to protect the vocal cords and subglottic space from aspiration of gastric contents or upper airway fluids, and can redirect such material safely into the esophagus. In some examples, the supraglottic umbrella 612 clears blood and other secretions from the hypopharynx/upper airway with removal.

FIG. 19B shows the endotracheal tube 600 of FIG. 19A inserted into the trachea 620 with the supraglottic umbrella 612 positioned near the proximal end of the trachea, where the glottis would be. The supraglottic umbrella 612 can also function to divert other tubes, devices, and secretions toward the esophagus, promoting passage into the esophagus and preventing unintended passage into the larynx and trachea with potential trauma.

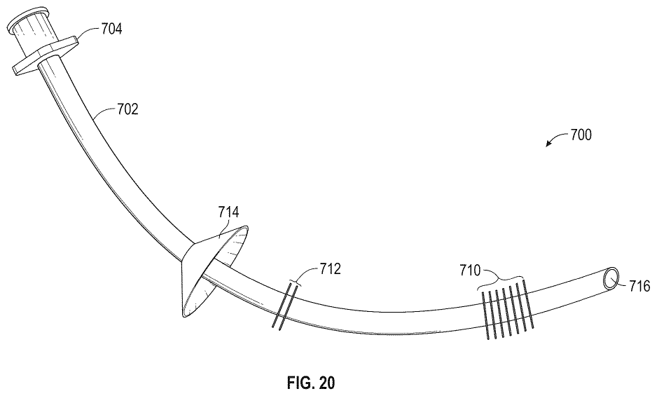

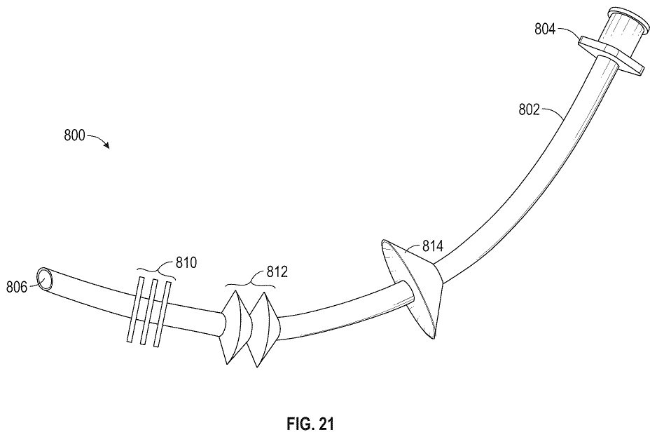

Similarly, FIG. 20 shows an exemplary endotracheal tube 700 including a tubular portion 702, a proximal adaptor 704, a distal opening 716, a distal set of baffles 710, a proximal set of baffles 712, and a supraglottic umbrella 714. The second set of baffles 712 can provide enhanced sealing, anchorage, and paratubular blockage.

FIG. 21 shows an exemplary endotracheal tube 800 including a tubular portion 802, a proximal adaptor 804, a distal opening 806, a distal set of baffles 810, a supraglottic umbrella 814, and an accordion-like expandable baffle portion 812 positioned between the distal baffles 810 and the supraglottic umbrella 814. The expandable baffle portion 812 can allow for adjustment in the axial distance between the distal opening 806, the baffles 810, and the supraglottic umbrella 814, to accommodate different sized anatomies and changes in tracheal dimensions with the respiratory cycle and head/neck movement. The expandable baffle portion 812 can be about the same diameter as the other baffles 810, or can be smaller or larger.

In some examples, an expandable baffle portion 812 can be positioned elsewhere along the tubular portion 802, such as proximal to the supraglottic umbrella 814 or between two sets of baffles. In some examples, two or more of such expandable baffle portions can be included. The extendibility of the tubular portion 802 can help accommodate changes in shape and can help prevent excessive pressure on the back of the throat that can result in mucosal erosions. Such extendable baffle portions can provide any range of extensibility, such as from a few millimeters to several centimeters (e.g., 1-2 centimeters).

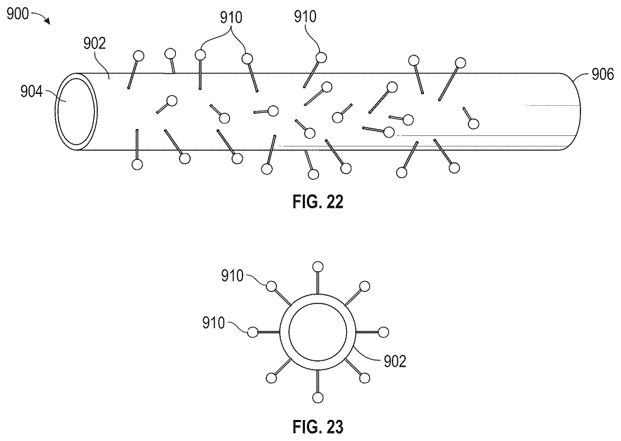

FIG. 22 shows an exemplary endotracheal tube 900 comprising a tubular portion 902, a distal opening 904, a proximal opening 906, and a plurality of discrete, flexible protuberances 910 extending radially from along the outer surface of the tubular portion 902. FIG. 23 shows an end view of the tubular portion 902 with an exemplary evenly spaced apart projection orientation. In other examples, the protuberances 910 can be more randomly spaced and randomly oriented along the tubular portion 902 to provide more even contact with the trachea and fewer and more tortuous flow pathways between the protuberances 910. While exemplary protuberances 910 are disclosed herein, in other examples, the protuberances 910 can be larger with a shorter stem, more or less occlusive (e.g., extending radially outward a different distance), and/or shaped or configured in other manners.

Like baffles, the protuberances 910 can provide sealing and/or limit migration. Such protuberances 910 can be combined with any one or more of the baffles described and illustrated herein in other examples. Thus, any of the baffled examples disclosed herein can be enhanced with added protuberances 910 along the tubular portion 902 adjacent to the baffles (e.g., proximal to, distal to, or between the baffles).

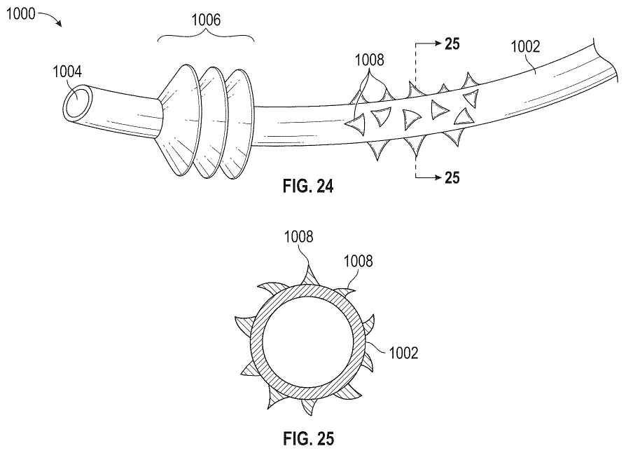

FIG. 24 shows an exemplary endotracheal tube 1000 including a tubular portion 1002, a distal opening 1004, a set of annular baffles 1006, and a plurality of discrete, flexible protuberances 1008 extending radially from along the outer surface of the tubular portion 1002. FIG. 25 shows an end view of the tubular portion 1002 with an exemplary orientation of protuberances 1008. The protuberances 1008 can have a curled or tilted terminus to aid in contacting the trachea. Like baffles 1006, the protuberances 1008 can provide sealing while also limiting migration and torsion. Any of the baffled examples disclosed herein can be enhanced with added protuberances 1008 along the tubular portion 1002 adjacent to the baffles 1006 (e.g., proximal to, distal to, or between the baffles).

The baffles of any of the baffled examples described and illustrated herein can comprise eccentric and/or non-continuous members that include circumferential spaces or gaps, which can help the baffles deform more readily and allow them to better conform to the shape of the trachea. For example, in some examples the baffles can be oval, egg-shaped, or other non-circular rounded shapes. In some examples, non-continuous baffle members can include one or more circumferential gaps, plural adjacent lobes, or an annular or semi-annular ring of protuberances or projections extending radially from the tubular portion.

The baffles of any of the baffled examples described and illustrated herein can help resist axial movement of the endotracheal tube from high ventilation pressures. In some examples, baffles angled proximally allow escape of high ventilation pressures and thereby reduce the risk of unwanted proximal migration of the endotracheal tube.

In some examples, the endotracheal tube prevents aspiration due to cuff deflation during performance of tracheostomy. In some examples, the baffles adjust their contour to maintain a seal with inspiration and expiration and accommodate expansion and contraction of the airway during the respiratory cycle. In some examples, the baffles can have a curled perimeter edge, which can help maintain orientation (e.g., prevent flipping of direction) with proximal and distal movement of the endotracheal tube.

In some examples, multiple baffles create closed compartments, which can limit biofilm formation on the distal part of the endotracheal tube. In some examples, multiple baffles create closed compartments between the baffles that prevent air escape, limit oxygen desaturation, and lessen the risk of airway fire during tracheostomy procedure. In some examples, the baffles minimize compression of the esophagus along the posterior tracheal wall, thereby avoiding dysphagia associated with inflated balloon cuffs. Some examples minimize effects on swallowing through spacing of baffles or specially shaped baffles. In some examples, the endotracheal tube with baffles has multiple projections or protuberances that allow torsion and flexibility, as described and illustrated in more detail above with reference to FIGS. 22-25, for example.

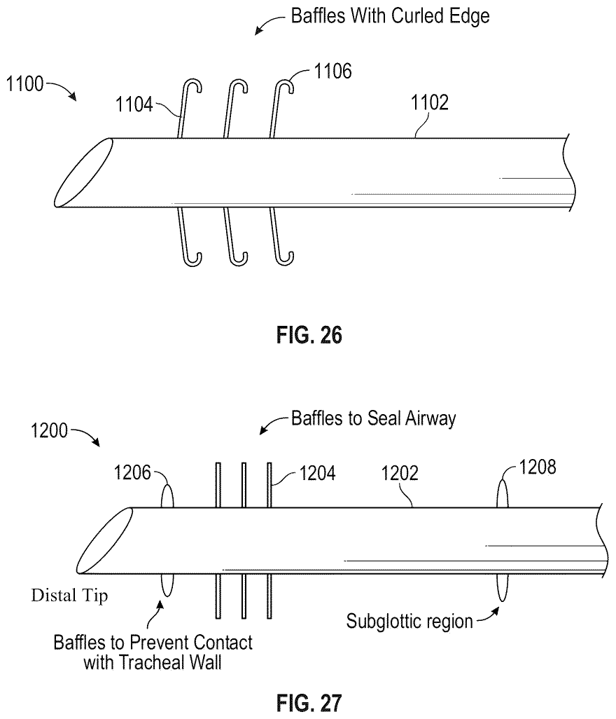

FIG. 26 shows an exemplary endotracheal tube 1100 that comprises a set of baffles 1104 that have curled edges 1106. In this example, the edges 1106 are curled proximally, while in other examples the edges 1106 of the baffles 1104 can be curled distally in a similar manner. The degree of the curling of the edges 1106 can vary. In some examples, the edges 1106 can curl around 90 degrees such that the edges 1106 point proximally. In other examples, the edges 1106 can curl around 180 degrees such that the edges 1106 point radially inwardly (similar to as shown in FIG. 26). In other examples, the edges 1106 of the baffles 1104 can be curled more than 180 degrees. In some examples, the entire annular peripheral edges 1106 of one or more of the baffle 1104 is curled, while in others only parts of the peripheral edges 1106 are curled.

FIG. 27 shows an exemplary endotracheal tube 1200 having a set of baffles 1204 that are broader to seal the airway, along with narrower proximal and distal baffles to prevent contact with the tracheal wall. A narrower distal baffle 1206 is positioned distal to the baffles 1204 nearer to the distal tip of the endotracheal tube 1200, and a narrower proximal baffle 1208 is positioned proximal to the baffles 1204 at the subglottic region. The narrower baffles help prevent the endotracheal tube 1200 from contacting the tracheal wall, such as when the endotracheal tube 1200 flexes or bends to conform to the native anatomy.

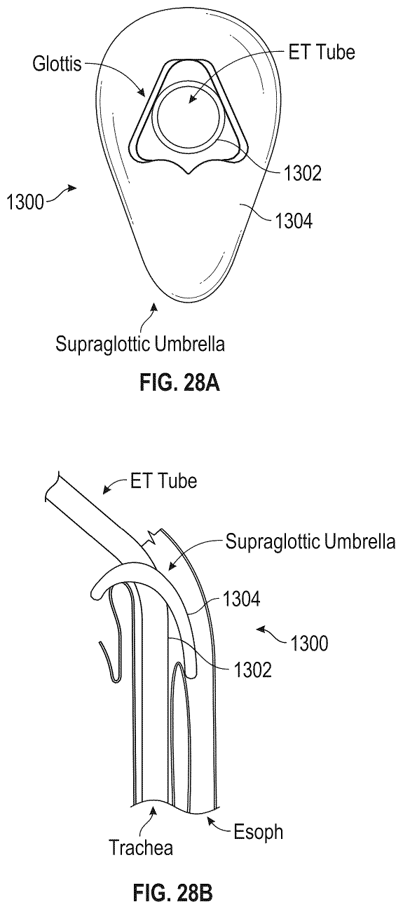

FIGS. 28A-B show an endotracheal tube 1300 having tubular portion 1302 and a supraglottic umbrella 1304 positioned over the glottis. The supraglottic umbrella 1304 extends radially from the tubular portion 1302, with the posterior aspect extending further than the rest of the supraglottic umbrella 1304 to extend toward and/or into the esophagus. As shown in the cross-sectional side view of FIG. 28B, the anterior aspect of the supraglottic umbrella 1304 can extend up and over and/or in front of the glottis, while the elongated posterior aspect can extend back and downwardly into the esophagus, creating a barrier to prevent materials (e.g., from the mouth or esophagus) from inadvertently entering the trachea while the endotracheal tube 1300 is in place, and vice versa. The supraglottic umbrella 1304 can also help standardize the distance of the distal end of the endotracheal tube 1300 from the glottis.

Since fluids can pool in the larynx, which are then at risk for aspiration, the supraglottic umbrella (e.g., of any of FIGS. 19-21 or 28A-B) can shield the larynx. For example, the supraglottic umbrella of any of the examples described and illustrated herein can be shaped like a semi-sphere or tapered anterior-posterior like a “fireman's hat” with a rear portion (e.g., a “duckbill” or “beavertail”). In some examples, the supraglottic umbrella includes a central longitudinal groove or concavity that directs feeding tubes posteriorly. Thus, the supraglottic umbrella can include a dedicated posterior groove to accommodate/direct a naso/orogastric feeding tube into the esophagus. The supraglottic umbrella can also be upturned upon insertion (e.g., windblown umbrella) and then be inverted in the opposite direction over the supraglottic larynx after the endotracheal tube is in position.

The supraglottic umbrella can vary in thickness and/or flexibility, with either the same or a different material as the associated endotracheal tube or other baffles included therewith. The supraglottic umbrella can be positioned more proximally on the tubular portion and then slid distally to contact the supraglottic larynx after the endotracheal tube is in position, thereby making the endotracheal tube depth adjustable. In yet other examples, the supraglottic umbrella can be separate from the tubular portion and attached after the endotracheal tube is in position.

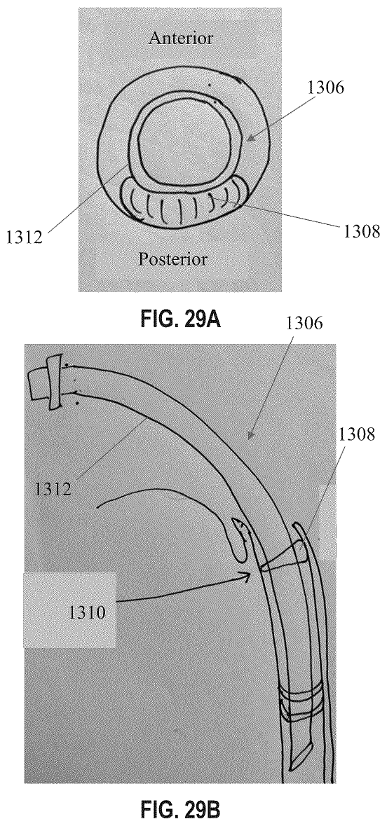

Referring to FIGS. 29A-B, an exemplary endotracheal tube 1306 with a projection 1308 proximate the glottis 1310. The projection 1308 in this example is a non-occlusive bumper, cushion, partial baffle, or other protuberance that prevents posterior contact of the tubular portion 1312 with the glottis 1310 and/or posterior subglottis due to the curvature of the tubular portion 1312 in that region. While the projection 1308 is located proximate the glottis 1310 in this example, the projection 1308 can be located elsewhere and/or any number of projections can be used in other examples to prevent contact of the tubular portion 1312 with the tracheal wall or laryngeal cartilages, for example.

Optionally, and as illustrated in FIGS. 29A-B, the projection 1308 can have a thicker posterior edge than an anterior edge. While the projection 1308 can be concentric with respect to the tubular portion 1312 in some examples (e.g., as described and illustrated above with reference to the distal baffle 1206 and proximal baffle 1208 of FIG. 27), the projection 1308 can also be disposed around only a portion of the circumference of the tubular portion 1312 at one or more locations. Additionally, the projection 1308 can be composed of a different material (e.g., a softer material) than the tubular portion 1312 in some examples.

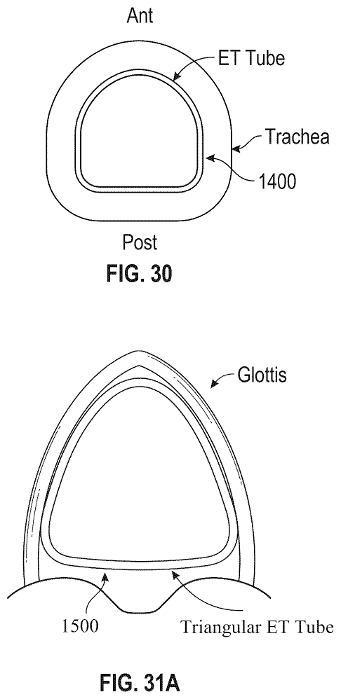

FIG. 30 shows an exemplary non-circular cross-sectional profile for an endotracheal tube 1400, with the posterior aspect having a flattened shape. The non-circular profile of the endotracheal tube 1400 can better match the native shape of the trachea/glottis. The anterior aspect is curved, while the posterior aspect is straight, or at least less curved than the anterior aspect. The lateral aspects of the profile can be curved or straight, or have an intermediate curvature that transitions from the curved anterior aspect to the flattened posterior aspect. The posterior-lateral aspects of the profile can include corners or more sharply curved aspects.

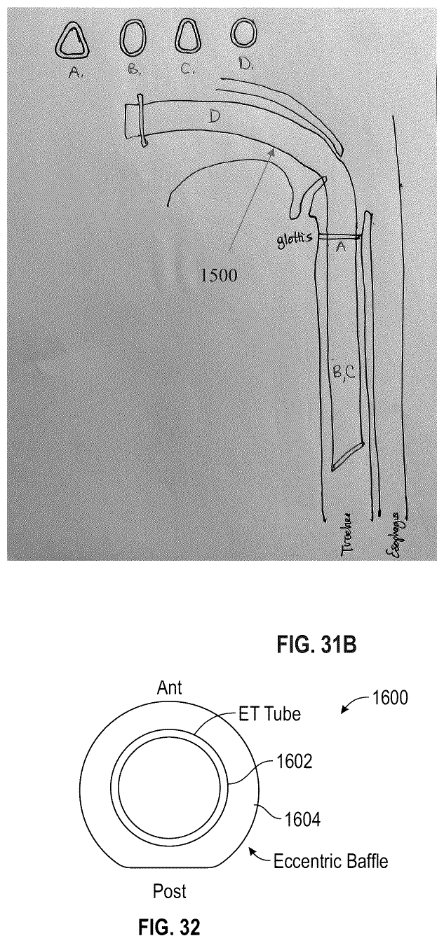

FIGS. 31A-B illustrate various exemplary endotracheal tube lumen shapes and cross-section profiles. Referring specifically to FIG. 31A, a generally triangular cross-sectional profile for an endotracheal tube 1500 is illustrated that is similar to the profile of the endotracheal tube 1400, but has a more triangular shape with three flattened sides or straighter aspects (e.g., the posterior aspect and two anterior-lateral aspects), and three corners or more sharply curved transitions between the three flattened sides, forming a generally triangular shape. This shape can better match the geometry of the glottis. Accordingly, since the endotracheal tube 1500 can exert pressure on the anterior glottis due to the triangular aperture, the lumen of the endotracheal tube 1500 can have a triangular shape or cross-section through at least a portion of the tubular portion, which also advantageously minimizes contact points with arytenoid cartilages

In other examples, the endotracheal tube 1500 can have an elliptical cross-sectional profile with a greater diameter in the anterior-posterior direction than in the lateral direction, which can better accommodate the native shape of the trachea, such as in the sub-glottic region (e.g., cricoid ring). In yet other examples, and since the endotracheal tube 1500 is in contact with the glottis and posterior subglottis due to curvature, a substantially straight section of the tubular portion can be provided in the subglottic region, a relatively small partial or complete baffle that prevents posterior contact can be provided on the endotracheal tube 1500, and/or the endotracheal tube 1500 lumen can have an oval or triangular shape or cross-section to minimize contact points with cricoid cartilage.

Accordingly, the tubular portion of the endotracheal tube 1500 can have a non-circular shape or cross-section at one or more portions, or the entirety of the tubular portion, to better accommodate the shape of the airway. Referring more specifically to FIG. 31B, the endotracheal tube 1500 in this example includes a generally circular shape (“D”) in the supraglottic region, a generally triangular shape (“A”) in the glottic region, and generally oval (“B”) and triangular (“C”) shapes in the subglottic and tracheal regions, although other permutations of these shapes, as well as other shapes, can be used for any region or portion of the lumen of the endotracheal tube 1500 in other examples.

Thus, the shape or cross-section profile of the tubular portion of the endotracheal tube 1500 could be uniform throughout or vary depending on corresponding region. The tubular portion could also vary in curvature to minimize pressure on contact points throughout or at a portion of the tubular portion. Other shapes or profiles, as well as combinations of those described and illustrated herein for different portions of an exemplary endotracheal tube 1500 can also be used in other examples.

FIG. 32 is a cross-sectional view of an exemplary endotracheal tube 1600 having a tubular portion 1602 and non-circular baffle 1604 that has a flat posterior aspect. In other examples, non-circular baffles can include two, three, or more flat or flattened aspects. In some examples, an endotracheal tube can include elliptical baffles, pear-shaped or egg-shaped baffles, and/or baffles of other non-circular shapes. Any of the baffle configurations disclosed herein may be oriented perpendicular to the endotracheal tube wall or non-perpendicular, such as projecting at an acute angle relative to the endotracheal tube wall.

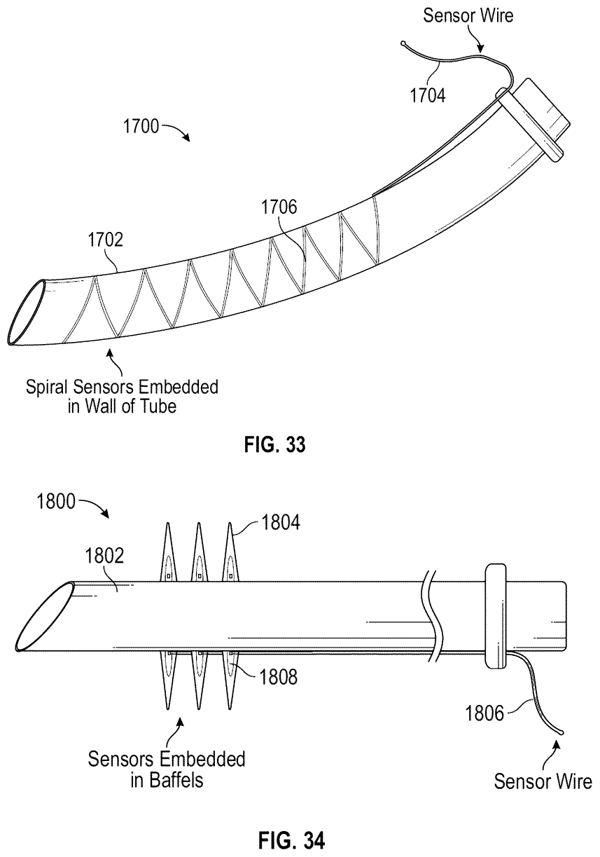

FIG. 33 shows an exemplary endotracheal tube 1700 that includes a tubular wall 1702, a sensor wire 1704, and a helical or spiral sensor 1706 embedded in the tubular wall 1702 and coupled to the sensor wire 1704. The spiral sensor 1706 can detect the presence of another object, such as an adjacent feeding tube, and/or can measure pressure, contact, position, deflection, movement, or other properties. The tube 1700 can also include one or more baffles in addition to the spiral sensor 1706 (e.g., any one or more of the baffles described and illustrated herein). The spiral sensors 1706 can also be used with integrated vocal cord nerve monitoring electrodes in some examples.

FIG. 34 shows an exemplary endotracheal tube 1800 that includes a tubular portion 1802, a set of baffles 1804, a sensor wire 1806, and sensors 1808 embedded in the baffles 1804 and coupled to the sensor wire 1806. The sensors 1808 can be annular, or partially annular, or can be present in a discrete array dispersed around the baffles 1804, or otherwise arranged within the baffles 1804. Such sensors 1808 can detect the presence of another object, such as an adjacent feeding tube, and/or can measure pressure, contact, position, deflection, movement, or other properties or parameters. For example, the sensors 1808 can detect directional deformation of the baffles.

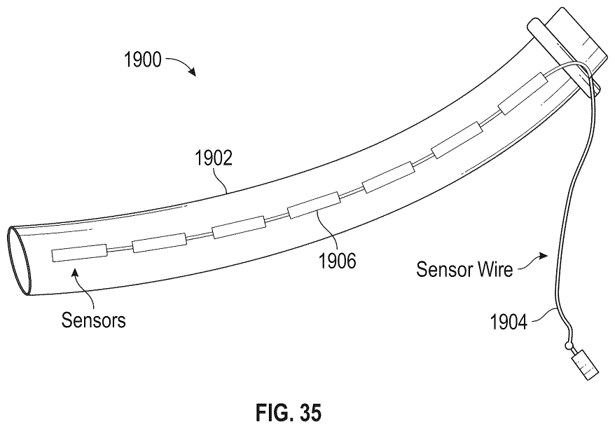

FIG. 35 shows an exemplary endotracheal tube 1900 that includes a tubular portion 1902, a sensor wire 1904, and a series of axially spaced apart sensors 1906 along the tubular portion 1902 and coupled to the sensor wire 1904. Such sensors 1906 can detect the presence of another object, such as an adjacent feeding tube, and/or can measure pressure, contact, position, deflection, movement, or other properties or parameters. The endotracheal tube 1900 can also include one or more baffles in addition to the sensor 1906 (e.g., any one or more of the baffles described and illustrated herein).

Thus, the sensors 1906 along the length of the endotracheal tube 1900, or a portion thereof, can identify contact of the endotracheal tube 1900 with the airway at multiple points and/or measure an amount of pressure. In addition to, or in place of, sensing pressure exerted on the endotracheal tube 1900 or baffles, sensors 1906 can detect changes in pH or other chemical changes, indicating episodes of aspiration, electrolyte levels, lactate levels, and/or end-tidal CO2 or other ventilated gas content. One or more of the sensors 1906 could be monitored by an attached ventilator unit or a separate physiologic monitoring system, for example, and other connectivity and/or monitoring topologies and/or methods can also be used in other examples.

In other examples, the sensor(s) of any of the examples described and illustrated herein with reference to FIGS. 33-35 can be configured to detect glottic movement and/or changes in pressure; deflection and/or orientation of baffles and/or movement of the endotracheal tube; directional movement of the endotracheal tube; and/or shape and/or deformation of endotracheal tube, and other characteristics and parameters can also be detected or measured in other examples.

Referring now to FIGS. 36A-C, an exemplary endotracheal tube 2000 with a tubular portion 2002 and a set of baffles 2004 with radiopaque indicators 2006 is illustrated. In these examples, radiopaque indicators 2006 are embedded in or deposited on one or more portions (e.g., the baffles 2004) of the endotracheal tube 2000. Referring specifically to FIG. 36A, a complete ring of radiopaque indicators 2006 is disposed around the outside edge of each of the baffles 2004. In FIG. 36B, the endotracheal tube 2000 includes the baffles 2004 with equidistant radiopaque indicators 2006 across each of the baffle 2004 and wrapping around each entire one of the baffles 2004 in a uniform line. In another example illustrated in FIG. 36C, the endotracheal tube 2000 includes baffles 2004 with radiopacity across the entire surface area of each of the baffles 2004.

In other examples, the radiopaque indicators 2006 could be a partial ring around the outside edge of one or more of the baffles 2004, any portion of one or more of the baffles 2004 could be infused with a radiopaque material, and/or one or more of the baffles 2004 could include points, symbols, designs, or any other type of deposition of radiopaque material on any complete or partial surface. In yet other examples, one or more of the baffles 2004 includes one of the radiopaque indicators 2006 at or near the midline of the baffle and/or at or near the base of the baffle. Accordingly, one or more of the baffles 2004 may have radiopaque indicators 2006 deposited after manufacturing and/or embedded during manufacturing, and the material itself of one or more of the baffles 2004 may be radiopaque in nature. In addition to, or in place of, position/depth, the radiopaque indicators 2006 on the baffles 2004 can indicate the direction of positioning of the baffles 2004 (e.g., flexed proximally or distally) in some examples.

While in the examples illustrated in FIGS. 36A-C, the radiopaque indicators 2006 are disposed at the baffles 2004, one or more portions of the tubular portion 2002, or another portion of the endotracheal tube 2000, can include radiopaque material in other examples. In some examples a series of radiopaque indicators are disposed along the length of the tubular portion 2002 and/or baffles 2004 for identification of depth and position of the endotracheal tube 2000. For example, the tubular portion 2002 may have one or more radiopaque indicators along a portion of its length, optionally with various notations of length according to the number and/or types of radiopaque indicators present. In another example, the diameter of the endotracheal tube 2000 may be identified by the combination and/or pattern of radiopaque indicators on one or more portions of the endotracheal tube 2000.

Accordingly, with this technology, when a patient is x-rayed, the clinicians will see how the baffles 2004 are fitting in the patient's airway as a result of the radiopaque indicators 2006. Thus, the radiopaque indicators 2006 add visual cues as to the sealing mechanism and efficacy to increase confidence in the insertion and placement of the endotracheal tube 2000. Additionally, the radiopaque indicators 2006 allow clinicians to analyze whether the size of the endotracheal tube 2000 is optimal for a particular patient.

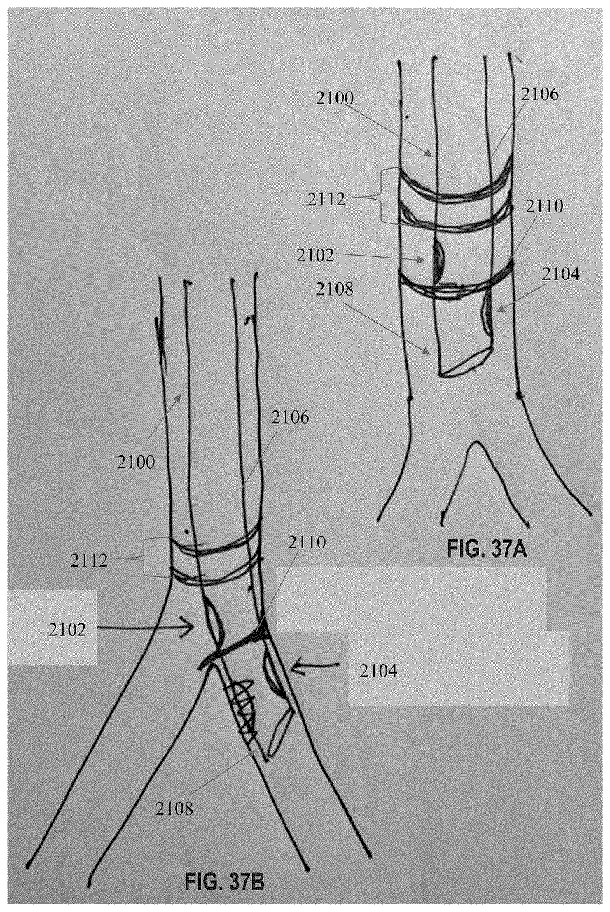

Referring to FIGS. 37A-B, an endotracheal tube 2100 with proximal and distal lateral openings 2102 and 2104 are illustrated. While radiopaque indicators can be used to analyze position of the endotracheal tube 2100, and identify distal migration, the proximal and distal lateral openings 2102 and 2104 in this example allow for continued ventilation of both lungs if there is distal migration of the endotracheal tube 2100 into the mainstem bronchus. Each of the proximal and distal lateral openings 2102 and 2104 are disposed in a wall of the tubular portion 2106 of the endotracheal tube 2100 toward a distal end 2108 of the tubular portion 2106, with the proximal lateral opening 2102 located proximally as compared to the distal lateral opening 2104.

Optionally, a distal baffle 2110 can be located between the proximal and distal lateral openings 2102 and 2104 in some examples. The distal baffle 2110 is a non-occlusive, partial, or smaller baffle located distally from any sealing baffles 2112 that may be coupled to the tubular portion 2106 of the endotracheal tube 2100. In other examples, more lateral opening can be disposed in the tubular portion 2106 in other locations.

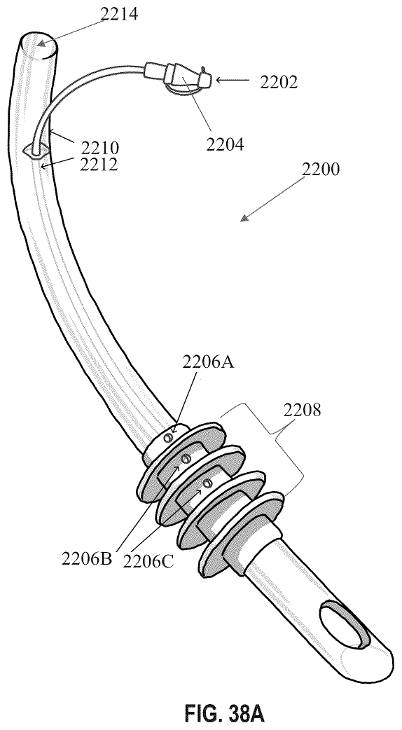

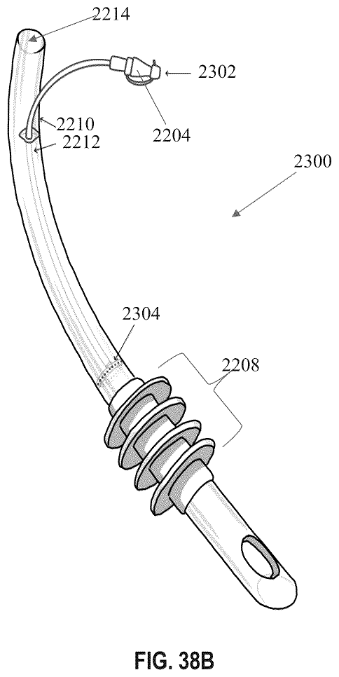

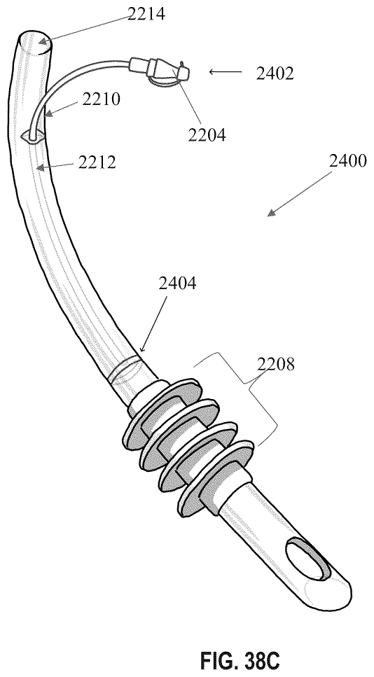

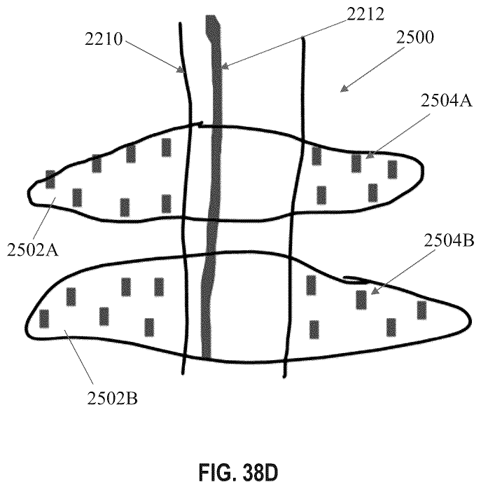

Referring now to FIGS. 38A-D, exemplary endotracheal tubes with exemplary port systems for suction and/or medication delivery are illustrated. The endotracheal tube 2200 in this example includes a port system 2202 that includes a port site 2204 and a plurality of multi-level ports 2206A-C at a distal end proximate a set of baffles 2208. In this example, the port 2206A is proximal to the most proximal baffle of the set of baffles 2208, which is a sealing baffle in this example, and the ports 2206B-C are each located between adjacent baffles of the set of baffles 2208, although any other number of ports in other locations and/or between any number of baffles can also be used in other examples.

The port site 2204 includes a lid and is external to a tubular portion 2210 of the endotracheal tube 2200. The port site 2204 is coupled to the ports 2206A-C via a cannula 2212 that transitions from external to the tubular portion 2210 to within the outer structure or outer wall of the tubular portion 2210 but not within the lumen 2214 of the tubular portion 2210 in this particular example. Accordingly, the port 2206A can include an opening that is in line with the base of the top or most proximal baffle of the set of baffles 2208. The endotracheal tube 2200 allows for a dedicated suction port system 2202 to get closer to the top of the sealing action (e.g., the most proximal baffle of the set of baffles 2208) and remove more secreted fluids where it pools.

With the port system 2202 that includes ports 2206A-C between baffles of the set of baffles 2208, as illustrated in FIG. 38A, each of the ports 2206A-C can have a neutral valve (not shown) that opens directly based on suction or blowing but remain neutral (closed) when no suction is attached (e.g., at the port site 2204). The valve can operate like a three section valve that is flexible in either direction with a neutral closed position, although other types of valves could also be used in other examples including two section valves. In some examples, the cannula 2212 includes a one-way valve to prevent backflow of medication (when the port system 2202 is used for medication delivery) or secretions (when port system 2202 for suction). Additionally, one or more of the ports 2206A-C can include an upward or proximal angle opening to prevent fluid from leaking to layers below (i.e., between other baffles of the set of baffles 2208).

Referring to FIG. 38B, another exemplary endotracheal tube 2300 with another exemplary port system 2302 for suction and/or medication delivery is illustrated. In this example, the port system 2302 includes a circumferential reservoir 2304 that includes a plurality of small (e.g., about 1 mm in diameter) pores located at a distal end of the cannula 2212. The circumferential reservoir 2304 is disposed around some or all of the tubular portion 2010 at a location proximal to the most proximal baffle in the set of baffles 2208, although other locations can also be used. When used for medication delivery, the pores of the circumferential reservoir 2304 seed the delivered medication slowly down the interior lumen 2214 of the tubular portion 2210.

Referring to FIG. 38C, another exemplary endotracheal tube 2400 with another exemplary port system 2402 for suction and/or medication delivery is illustrated. In this example, the port system 2402 includes another circumferential reservoir 2404 without the pores of the circumferential reservoir 2304 illustrated in FIG. 38B. The circumferential reservoir 2404 also is disposed around some or all of the tubular portion 2010 at a location proximal to the most proximal baffle in the set of baffles 2208, although other locations can also be used. The circumferential reservoir 2404 is a relatively thin opening that allows for injections to flow down the lumen 2214 of the tubular portion 2010 during medication delivery.

Referring to FIG. 38D, another exemplary endotracheal tube 2500 with another exemplary port system for suction and/or medication delivery is illustrated. In this example, the port system includes the cannula 2212 coupled to baffles 2502A-B, and specifically a plurality of ports 2504A-B disposed proximate the baffles 2502A-B, respectively. The baffles 2502A-B can be hollow and/or includes relatively small channels that connect the ports 2504A-B to the cannula 2212, for example, although other arrangements of components can also be used. The ports 2504A-B and port system can be configured to drain to a central channel as illustrated in FIGS. 38A-C in some examples.

In other examples, the distal injection ports can be located elsewhere, including toward a proximal end of the endotracheal tube 2200, such as proximal to a supraglottic umbrella (e.g., as illustrated in FIGS. 19-21 and 28). In yet other examples, any combination of the ports illustrated in FIGS. 38A-D can be used. Additionally, any of the port systems illustrated in FIGS. 38A-D can be used for intra-airway medication (e.g., liquid or aerosol) delivery without breaking the seal of the ventilatory circuit when at least one of the ports is located distal to the most distal baffle of the set of baffles 2208. This most distal port can also have a one-way reflux valve that prevents air leak or medication reflux.

The following features can also be included in any of the exemplary baffled endotracheal tube examples described and illustrated herein: a lumen with a triangular cross-sectional shape at the level of the glottis to minimize traumatic injury to the glottic larynx; elliptical baffles angled to facilitate insertion through the glottis by entering the posterior glottis first and displacing the vocal cords from posterior to anterior as the baffle passes; spacing of baffles to prevent mucosal injury to subglottic mucosa associated with prolonged intubation; a baffle shape that prevents accidental passage of nasogastric or orogastric feeding tubes into the airway; a supraglottic umbrella that anchors the endotracheal tube superiorly, assuring that the endotracheal tube is fully inserted and preventing contact of tip with carina or distal insertion into the mainstem bronchus (especially in women); a position of the supraglottic umbrella that is adjustable to vary the length of the endotracheal tube distal to the glottis; an elliptical shape that fits the contour of the subglottic region better, thereby providing a maximal airway without contact with subglottic mucosa; a spiral baffle design that allows greater contact of nerve monitoring electrodes with vocal cords; a triangular lumen that maintains orientation of the endotracheal tube within the trachea and minimizes trauma to glottis; a proximal set of baffles that anchors the endotracheal tube at the level of the glottis and prevents pooling of secretions in the subglottic airway; a design of the baffles that alters the maximal ventilation pressure at which leakage of air occurs; variations in shape, thickness, and/or stiffness that minimizes pressure exerted on the esophagus posteriorly to limit dysphagia associated with cuffed endotracheal tubes; and/or proximal glottic baffles at varying distance from distal baffles that allow accurate placement of endotracheal tube at an optimal distance from the carina and subglottic region.

Benefits of the disclosed baffled endotracheal tubes of the examples described and illustrated herein can include any of the following: prevention of aspiration and ventilator-associated tracheobronchitis/pneumonia; prevention of distal and proximal tube migration; avoidance of risks of pressure necrosis from high cuff pressures; prevention of tracheal stenosis; avoidance of complications from continuous subglottic suction; prevention of aspiration of gastric contents; prevention of tube migration due to airway movement associated with the respiratory cycle or changes in head/neck position; safer tracheostomy procedure; and/or any other benefits or advantages disclosed elsewhere herein, in any combination.

Baffled tube designs analogous to those described elsewhere herein can also be used for tracheostomy tubes, with similar features and advantages. Exemplary benefits of a tracheostomy tube with baffles include any one or more of the following: easier insertion and removal; prevention of aspiration and ventilator-associated tracheobronchitis/pneumonia; prevention of distal and proximal tracheostomy tube migration; avoid risks of pressure necrosis from high cuff pressures; and avoid rupture of cuff during insertion and cuff malfunction.

In addition to the other benefits described herein, other benefits can be provided by tracheostomy tubes with baffles. In some tracheostomy tubes with baffles, internal and external baffles at the stoma prevent accidental decannulation. In some tracheostomy tubes with baffles, a spiral baffle allows phonation. In some tracheostomy tubes with baffles, baffles center the tracheostomy tube within the tracheal lumen and prevent impingement on the tracheal wall.

Baffled tube designs analogous to those described elsewhere herein can also be used for esophageal stents, with similar features and advantages. For example, esophageal stents with baffles can help to prevent migration of the esophageal stent. In some examples, esophageal stents with baffles can include a continuous helical or spiral baffle. Compared to examples with discrete separated baffles, a continuous spiral baffle can allow secretions and other materials to pass without stasis and can avoid focal circumferential pressure on the esophagus.

Some esophageal stent examples can include multiple independent protuberances, projections, stalks, or bristles that anchor the tube but allow greater flexibility (in addition to baffles, or instead of baffles). Exemplary benefits of esophageal stents with baffles can include: prevention of proximal and distal migration; avoid complications associated with suturing of esophageal stent; and more effective sealing of luminal perforation.

Characteristics, materials, and other features described in conjunction with a particular aspect or example(s) of the disclosed technology are to be understood to be applicable to any other aspect or example(s) described herein unless incompatible therewith. Thus, any of the features described and illustrated herein may be combined in any combination, except combinations where at least some of such features are mutually exclusive. This technology is not restricted to the details of any foregoing examples and extends to any one, or any combination, of the features disclosed herein.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element. As used herein, the term “and/or” used between the last two of a list of elements means any one or more of the listed elements. For example, the phrase “A, B, and/or C” means “A”, “B,”, or “C”, “A and B”, “A and C”, “B and C”, or “A, B, and C.” As used herein, the term “coupled” generally means physically coupled or linked and does not exclude the presence of intermediate elements between the coupled items absent specific contrary language.

In view of the many possible examples to which the principles of the disclosed technology may be applied, it should be recognized that the illustrated examples are only examples and should not be taken as limiting the scope of the disclosure. Rather, the scope of the disclosure is at least as broad as the full scope of the following claims.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

1. An endotracheal tube, comprising:
a tubular portion;
a proximal adaptor extending from a proximal end of the tubular portion;
a distal opening at or adjacent to a distal end of the tubular portion; and
one or more baffles positioned around the tubular portion between the proximal adaptor and the distal opening, wherein the baffles are formed separately from, and coupled to, the tubular portion and at least one of the baffles is configured to contact an inner wall of a trachea when the endotracheal tube is inserted into the trachea to resist migration of the endotracheal tube relative to the trachea and reduce aspiration between the tubular portion and the inner wall of the trachea.

2. The endotracheal tube of claim 1, wherein one or more of the baffles are helical, annular, angled proximally or distally from the tubular portion, have a flattened posterior aspect, have a curled perimeter edge, comprise a wavy cross-sectional profile having edges configured to interface with the trachea and fold distally to thereby reduce aspiration, or extend perpendicularly from the tubular portion.

3-4. (canceled)

5. The endotracheal tube of claim 1, wherein one or more of the baffles comprise a material that is resiliently deformable and conforms to one or more contours of the trachea.

6-8. (canceled)

9. The endotracheal tube of claim 1, wherein the one or more baffles comprise a plurality of individual baffles that each has a base portion extending from the tubular portion, a first angled portion extending proximally from the base portion, and a second angle portion extending distally from the base portion.

10. The endotracheal tube of claim 1, further comprising a loading tube that is positioned around the one or more baffles before the one or more baffles are inserted into the trachea and is configured to be retracted off of the one or more baffles after the one or more baffles are positioned within the trachea.

11. The endotracheal tube of claim 1, further comprising a supraglottic umbrella positioned around, and extending radially from, the tubular portion proximal to the one or more baffles, wherein the supraglottic umbrella is configured to block aspiration of materials into the trachea.

12. (canceled)

13. The endotracheal tube of claim 11, wherein the supraglottic umbrella has a posterior portion that extends to a greater radial distance from the tubular portion than an anterior portion of the supraglottic umbrella, such that the posterior portion can extend over or into an esophagus.

14. The endotracheal tube of claim 1, further comprising an axially expandable baffle portion positioned proximal to the one or more baffles, wherein the axially expandable baffle portion has a greater diameter than the tubular portion.

15. The endotracheal tube of claim 1, wherein the one or more baffles comprise a first material and the tubular portion comprises a second material that is different than the first material.

16. The endotracheal tube of claim 1, wherein the tubular portion or the one or more baffles comprises reinforcing elements in selected regions to provide asymmetric reinforcement.

17. The endotracheal tube of claim 1, wherein the tubular portion forks into two forks at the distal end, wherein each fork has another one or more baffles.

18. (canceled)

19. The endotracheal tube of claim 1, wherein the tubular portion has a non-circular cross-sectional profile in at least one region and the non-circular cross-sectional profile comprises a flattened posterior aspect, a triangular cross-sectional profile, or an elliptical cross-sectional profile.

20. (canceled)

21. The endotracheal tube of claim 1, further comprising at least one sensor embedded in or coupled to the tubular portion or one or more of the baffles, wherein the at least one sensor comprises a helical sensor or a series of sensors axially spaced apart along the tubular portion and is configured to measure one or more of pressure, contact, position, deflection, or movement of one or more portions of the endotracheal tube.

22-23. (canceled)

24. The endotracheal tube of claim 1, further comprising:
a port system for suction or medication delivery, wherein the port system comprises a port site coupled to a proximal portion of a cannula that extends from external to the tubular portion at the proximal portion of the cannula to along and integral with a wall of the tubular portion, without entering a lumen of the tubular portion, toward a distal portion of the cannula; and
at least one port adjacent to, or disposed within, one or more of the baffles and fluidly coupled to the cannula and the port site.

25. The endotracheal tube of claim 24, wherein the one or more baffles comprise a plurality of baffles and at least one of:
the port comprises a plurality of ports, and at least one of the ports is disposed between two of the plurality of baffles; or
the port is proximal to a most-proximal one of the plurality of baffles or distal to a most-distal one of the plurality of baffles.

26. (canceled)

27. The endotracheal tube of claim 1, wherein one or more of at least a portion of the tubular portion or one or more of the baffles is impregnated or coated with a substance, wherein the substance comprises an anti-fouling material or a flame-retardant material.

28. (canceled)

29. The endotracheal tube of claim 1, wherein:
one or more of the baffles comprise a plurality of portions comprising at least interior and exterior portions each formed of different materials having different properties; and
the inner portion is closer to the tubular portion than the exterior portion and is less pliable than the exterior portion.

30. The endotracheal tube of claim 1, further comprising a projection disposed proximal to the baffles and configured to be disposed adjacent to a glottis when the endotracheal tube is inserted into the trachea, wherein the projection is non-occlusive and comprises a posterior edge that is thicker than an anterior edge of the projection.

31. The endotracheal tube of claim 1, further comprising one or more radiopaque indicators embedded in or deposited on one or more of the tubular portion or one or more of the baffles.

32. (canceled)

33. The endotracheal tube of claim 1, further comprising a distal lateral opening in the tubular portion and a proximal lateral opening in the tubular portion, wherein the distal lateral opening is disposed further toward the distal end of the tubular portion than the proximal lateral opening and the baffles further comprise a distal baffle that is non-occlusive and positioned around the tubular portion between the distal and proximal lateral openings.
